(19) United States
(12) Patent Application Publication
(10) Pub. No.: US 2004/0228884 A1
(43) Pub. Date: Nov. 18, 2004

(54) Ion-pair delivery system for cosmetic and pharmaceutical compositions

(76) Inventor: Shyam K. Gupta, Scottsdale, AZ (US)

Correspondence Address: SHYAM K. GUPTA, BIODERM RESEARCH, 5221 E. WINDROSE DRIVE, SCOTTSDALE, AZ, 85254, US

(21) Appl. No.: 10/439,349

(22) Filed: May 15, 2003

(51) Int. Cl. A61K 31/665; A61K 7/00; A61K 31/19

(52) U.S. Cl. 424/401; 514/100; 514/557; 514/574; 514/474

(57) ABSTRACT

This invention relates to a novel ion-pair delivery system useful for cosmetic, pharmaceutical, and topical nutraceutical applications in which the functional performance and consumer aesthetics of an electron donor composition and an electron acceptor composition, or a proton donor composition and a proton acceptor composition, are synergistically enhanced when such compositions are combined in an ion-pair mode. During ion-pair bonding process, the electron donor composition or the proton acceptor composition become positively charged and the electron acceptor composition or proton donor composition become negatively charged and thus bind together in an ionic manner. Such ion-pair compositions release their electronically bound components in their original state when such compositions are absorbed into skin and reach physiological pH conditions.

### CROSS-REFERENCE TO RELATED APPLICATIONS

Not Applicable.

### STATEMENT REGARDING FEDERALLY SPONSORED RESEARCH OR DEVELOPMENT

Not Applicable.

### REFERENCE TO SEQUENCE LISTING, A TABLE, OR A COMPUTER PROGRAM LISTING COMPACT DISK APPENDIX

Not Applicable.

### BACKGROUND OF INVENTION

It is well recognized in the scientific community that delivery systems are highly useful in cosmetics and pharmaceutical disciplines. The search for new delivery systems now incorporates multi-disciplinary fields encompassing chemistry, biology, medicine, and package engineering. A cosmetic delivery system is a composition or process that can enhance perceptual or measured performance of a cosmetic product. A delivery system is thus a combination of both art and science that can improve the performance and consumer appeal of a consumer product or composition. In a recent article written by present inventor (Cosmetic Delivery Systems, Household & Personal Products Industry, commonly known as HAPPI magazine, January 2003 issue, page 79) the definition and benefits of a number of prior art delivery systems have been discussed. Additional delivery systems have been described in a recent text book on this subject (S. Magdassi and E. Touitou, eds., Novel Cosmetic Delivery Systems, Marcel Dekker, N.Y., 1998). The design of a delivery system can encompass a combination of desirable product attributes that include ingredient(s) and composition(s) themselves, formulation, manufacturing process, packaging, performance, sensory attributes, efficacy, deposition, bioavailability, synergism, and compositional stability. A number of currently popular delivery systems include Adsorption/Entrapment methods (that are based on various Clays, Polystyrene, Fumed Silica, Metal Oxides, Zeolites, Dextrins, Polyamides, Nylon, Microcapsules, Nanoparticles, and Thin Films), Emulsion methods (that includes Hydrogels and Lipogels), Enzymes (for skin exfoliation), Suspension methods (that includes Anhydrous Systems), Vesicles (that includes Liposomes, Nanosomes, and Hollow Micro-beads), and lonotropic methods (that are based on diffusion by weak potential electric current).

A number of cosmetic and pharmaceutical compositions are available that are poorly bioavailable in their original state due to their poor dissolution rate, bioavailability, consumer appeal, or stability problems. In the prior art, such problems have been partially solved by the preparation of certain solubility enhancing derivatives.

For example, lipoic acid (a very popular antioxidant) is practically insoluble in water, but sodium lipoate; made by the reaction of lipoic acid with sodium hydroxide (as shown in Equation 1) is soluble in water.

Lipoic Acid+NaOH→Na Lipoate+H₂0 (Equation 1)

Ascorbic acid, although soluble in water, has poor stability in water solutions in the presence of air. However, calcium ascorbate, made by the reaction of ascorbic acid with calcium carbonate (Equation 2), has both water solubility and better stability in such water solutions.

2 Ascorbic acid+CaCO₃→Ca(Ascorbate)₂+CO₂+H₂O (Equation 2)

However, as can be noted above, the attachment of other ions to such molecules (for example, sodium ion to lipoic acid and calcium ion to ascorbic acid) also reduces their skin care benefits due to their molecular weight dilution. In the molecular weight dilution, the composition that is attached to another molecule does not have any skin or body beneficial benefit that is complementary to original molecule or composition. In the examples mentioned in Equation 1, sodium does not contribute anything toward the antioxidant benefit of lipoic acid. In fact, sodium increases the molecular weight of lipoic acid when it is converted into sodium lipoate. This results in increased weight requirement for sodium lipoate (compared to the weight needed for lipoic acid itself) to get the same antioxidant benefits of alpha-lipoic acid. In Equation 2, calcium ion similarly increases the molecular weight of ascorbic acid, but does not contribute benefits toward the collagen synthesis boosting effect of ascorbic acid.

It would thus be highly desirable if the bioavailability, consumer appeal, or stability, and such of cosmetic and pharmaceutical compositions can be improved by the prior art methods shown in Equation 1 and Equation 2, but in which both moieties of such compositions are beneficial in a synergistic manner.

Surprisingly, I have now found that the combination of a cosmetic or pharmaceutical composition by using another cosmetic or pharmaceutical composition by an ion-pair mechanism, as shown in Equation 3, solves this problem. The ion-pair combinations thus formed are more bioavailable, have better stability, and are economical to produce from commonly available ingredients.

Electron Donor Composition+Electron Acceptor Composition→Ion-Pair Composition (Equation 3)

For example, lactic acid is well known for its skin cells turnover rate enhancement benefits. However, it is also very irritating to skin, hence of poor consumer appeal. The combination of lactic acid with sodium hydroxide gives sodium lactate. Sodium lactate is much less irritating to skin, hence more consumer acceptable. However, bioavailability of sodium lactate is very poor, as it does not penetrate through the skin easily and is not rapidly absorbed into skin. Another ingredient, Niacinamide, is also well known for its skin beneficial properties, including the enhancement of skin cell growth. The combination of lactic acid, which is an electron-acceptor or proton-donor, with niacinamide, which is an electron-donor or proton-acceptor, forms niacinamide lactate, which is an ion-pair (Equation 4). However, this ion-pair of niacinamide lactate is more bioavailable and easily absorbed through skin without causing any skin irritation. Moreover, the skin beneficial efficacy of both niacinamide and lactic acid are enhanced in a synergistic manner.

Lactic acid+Niacinamide→Niacinamide Lactate (Equation 4)

In another example, ascorbic acid, which is an electron acceptor, is combined in an ion-pair mode with glucosamine, which is an electron donor, to form glucosamine ascorbate (Equation 5). The collagen boosting benefits of both glucosamine and ascorbic acid are thus combined, which, additionally, also provides their enhanced bioavailability, improved stability in water solutions in the presence of air, and synergistically increased collagen synthesis benefits.

Ascorbic acid+Glucosamine→Glucosamine Ascorbate (Equation 5)

Although not bound by any theory or hypothesis, it is my belief that the ion-pair delivery system of the present invention works because the ion-pair undergoes ion-separation under the conditions of physiological pH (which is about 7.4), when such compositions are absorbed into skin, as shown in Equation 6.

Ion-Pair Composition→Electron Donor Composition+Electron Acceptor Composition (Equation 6)

Such ion-separation of ion-pair composition releases the corresponding compositions in their active form and at the site of their action. For example, Glucosamine ascorbate undergoes ion-separation under the conditions of physiological pH to release both glucosamine and ascorbic acid in their original electronic states, as shown in Equation 7.

Glucosamine Ascorbate→Glucosamine+Ascorbic Acid (Equation 7)

Such separation of ion-pairs formed by metal ions is not known to occur under the conditions of physiological pH. For example, Sodium Lipoate is not released into sodium hydroxide and lipoic acid molecules under the conditions of physiological pH. This is one of the reasons for the poor bioavailability of such metal atom based ion-pairs.

Regardless of the actual mechanism of the benefits caused by the ion-pair compositions prepared according to present invention, the benefits of such ion-pair delivery system are unprecedented in the prior art.

### SUMMARY OF INVENTION

This invention relates to a novel ion-pair delivery system useful for cosmetic, pharmaceutical, and topical nutraceutical applications in which the functional performance and consumer aesthetics of an electron donor composition and an electron acceptor composition, or a proton donor composition and a proton acceptor composition, are synergistically enhanced when such compositions are combined in an ion-pair mode. During ion-pair bonding process, the electron donor composition or the proton acceptor composition becomes positively charged and the electron acceptor composition or proton donor composition becomes negatively charged, and thus they both bind together in an ionic manner. Such ion-pair compositions release their electronically bound components in their original state when such compositions are absorbed into skin and reach physiological pH conditions.

### DETAILED DESCRIPTION

This invention describes the combination of a cosmetic or pharmaceutical composition by using another cosmetic or pharmaceutical composition by an ion-pair mechanism in which one composition is an electron-donor or proton-acceptor, and the other composition is an electro-acceptor or proton-donor to form the ion-pair combinations that are more bioavailable, have better stability, and are economical to produce from commonly available ingredients.

The ion-pair compositions thus formed are not just the mixtures of the two compositions that are combined in an ion-pair mode to form such ion-pair compositions. Such ion-pair compositions are discreet chemical entities. For example, the combination of niacinamide with ascorbic acid results in the formation of ion-pair complex, niacinamide ascorbate. In this example, niacinamide, which is a proton-acceptor composition, has the following properties: crystalline white powder, melting point 130° C., pH of 1% water solution=6.3, solubility in water=50%, stability of water solution=good. Ascorbic acid, which is a proton-donor composition, has the following properties: crystalline white powder, melting point 162° C., pH of 1% water solution=2.6, solubility in water=25%, stability of water solution=poor. Niacinamide ascorbate, the ion-pair that is produced from the combination of the above two compositions has the following properties: crystalline yellow powder, melting point 142° C., pH of 1% water solution=3.7, solubility in water=40%, stability of water solution=good. Niacinamide ascorbate is rapidly absorbed into skin from water solutions, and upon reaching physiological pH conditions it undergoes ionic separation to release both niacinamide and ascorbic acid in their original molecular state.

Ascorbic acid is known to be a problematic ingredient in cosmetic compositions from its stability and bioavailability point of view. However, the ion-pair compositions formed from the ion-pair combination of ascorbic acid with appropriate electron-donor compositions are more stable and are easier to formulate in such cosmetic compositions. The examples of such ion-pair compositions of ascorbic acid include, but not limited to, glucosamine ascorbate, arginine ascorbate, lysine ascorbate, glutathione ascorbate, nicotinamide ascorbate, niacin ascorbate, allantoin ascorbate, creatine ascorbate, creatinine ascorbate, chondroitin ascorbate, chitosan ascorbate, DNA Ascorbate, and carnosine ascorbate.

Hydroxycitric acid is a popular composition for weight loss management. However, it is unstable in its free acid form and is known to undergo cyclization reaction to form Garcinia acid, which does not provide weight loss benefits. Most preparations of hydroxycitric acid are thus based on its alkali and alkaline earth metal salts, such as tri-potassium hydroxycitrate and calcium hydroxycitrate. In such compositions, the tri-potassium part or calcium part does not provide any weight loss benefits. However, by ion-pair combination of hydroxycitric acid with niacinamide, niacinamide hydroxycitrate ion-pair is obtained in which both hydroxycitric acid part and niacinamide part provide slimming benefits. Moreover, in Niacinamide hydroxycitrate, hydroxycitric acid part is not cyclized to Garcinia acid form. Additional skin beneficial ion-pair compositions of hydroxycitric acid (HCA) can also be prepared, for example Allantoin HCA, Glucosamine HCA, Creatine HCA, Carnitine HCA, Niacinamide HCA, Pyridoxine HCA, Chitosan HCA, Niacin HCA, Benzyl Niacin HCA, Methyl Niacin HCA, Caffeine HCA, Aminophylline HCA, Chromium picolinate HCA, Phaseolamin HCA, Theophylline HCA, Theobromine HCA, Synephrine HCA, Hordenine HCA, Octopamine HCA, Tyramine HCA, and N-Methyltyramine HCA, and such.

AHA (alpha-hydroxy acids) and BHA (beta-hydroxy acids) are very popular cosmetic compositions that provide skin rejuvenating benefits. However, such acids are known to cause skin irritation. If they are neutralized with an alkali, for example, then their skin irritation is lowered, but their skin rejuvenating efficacy is also significantly reduced or even eliminated. The combination of AHA or BHA with proton-accepting compositions eliminates these problems, and such ion-pair compositions, for example allantoin lactate, allantoin glycolate, allantoin mandelate, allantoin malate, allantoin ascorbate, allantoin phytate, allantoin citrate, allantoin hydroxy citrate, allantoin aleurate, allantoin salicylate, allantoin hyaluronate, glucosamine lactate, glucosamine glycolate, glucosamine malate, glucosamine mandelate, glucosamine ascorbate, glucosamine phytate, glucosamine citrate, glucosamine hydroxy citrate, glucosamine aleurate, glucosamine salicylate, glucosamine hyaluronate, creatine lactate, creatine glycolate, creatine malate, creatine mandelate, creatine ascorbate, creatine phytate, creatine citrate, creatine hydroxy citrate, creatine aleurate, creatine salicylate, creatine hyaluronate, niacinamide lactate, niacinamide glycolate, niacinamide malate, niacinamide mandelate, niacinamide ascorbate, niacinamide phytate, niacinamide citrate, niacinamide hydroxy citrate, niacinamide aleurate, niacinamide salicylate, niacinamide hyaluronate, pyridoxine lactate, pyridoxine glycolate, pyridoxine malate, pyridoxine mandelate, pyridoxine ascorbate, pyridoxine phytate, pyridoxine citrate, pyridoxine hydroxy citrate, pyridoxine aieurate, pyridoxine salicylate, pyridoxine hyaluronate, chitosan lactate, chitosan glycolate, chitosan malate, chitosan mandelate, chitosan ascorbate, chitosan phytate, chitosan citrate, chitosan hydroxy citrate, chitosan aleurate, chitosan salicylate, and chitosan hyaluronate can be composed for their various skin and body beneficial applications.

Several acne compositions are already known. Such compositions can be further enhanced in their efficacy and bioavailability by ion-pair combination process described in the present invention. The examples of such acne compositions include, but not limited to, niacinamide salicylate, niacinamide ascorbate, niacinamide folate, niacinamide lipoate, niacinamide lactate, niacinamide glycolate, niacinamide mandalate, niacinamide malate, niacinamide hydroxycitrate, niacinamide hydroxytetronate, niacinamide aleurate, niacinamide petroselinate, niacinamide pantothenate, niacinamide adenosine monophosphate (AMP), niacinamide diphosphate (ADP), niacinamide adenosine triphosphate (ATP), niacinamide hydroquinone carboxylate, allantoin lactate, allantoin glycolate, allantoin mandelate, allantoin malate, allantoin ascorbate, allantoin phytate, allantoin citrate, allantoin hydroxy citrate, allantoin aleurate, allantoin salicylate, allantoin hyaluronate, glucosamine lactate, glucosamine glycolate, glucosamine malate, glucosamine mandelate, glucosamine ascorbate, glucosamine phytate, glucosamine citrate, glucosamine hydroxy citrate, glucosamine aleurate, glucosamine salicylate, glucosamine hyaluronate, creatine lactate, creatine glycolate, creatine malate, creatine mandelate, creatine ascorbate, creatine phytate, creatine citrate, creatine hydroxy citrate, creatine aleurate, creatine salicylate, creatine hyaluronate, niacinamide lactate, niacinamide glycolate, niacinamide malate, niacinamide mandelate, niacinamide ascorbate, niacinamide phytate, niacinamide citrate, niacinamide hydroxy citrate, niacinamide aleurate, niacinamide salicylate, niacinamide hyaluronate, pyridoxine lactate, pyridoxine glycolate, pyridoxine malate, pyridoxine mandelate, pyridoxine ascorbate, pyridoxine phytate, pyridoxine citrate, pyridoxine hydroxy citrate, pyridoxine aleurate, pyridoxine salicylate, pyridoxine hyaluronate, chitosan lactate, chitosan glycolate, chitosan malate, chitosan mandelate, chitosan ascorbate, chitosan phytate, chitosan citrate, chitosan hydroxy citrate, chitosan aleurate, chitosan salicylate, chitosan hyaluronate, azelaic acid, niacinamide azelate, pyridoxine azelate, chitosan azelate, glucosamine azelate, retinoic acid, niacinamide retinoate, pyridoxine retinoate, chitosan retinoate, and glucosamine retinoate, and combinations thereof.

Niacinamide is a popular cosmeceutical ingredient. The ion-pair compositions of niacinamide that are useful for various skin, hair, and body beneficial synergistic compositions include, but not limited to niacinamide salicylate, niacinamide ascorbate, niacinamide folate, niacinamide lipoate, niacinamide lactate, niacinamide glycolate, niacinamide mandalate, niacinamide malate, niacinamide hydroxycitrate, niacinamide hydroxytetronate, niacinamide aleurate, niacinamide petroselinate, niacinamide pantothenate, niacinamide adenosine monophosphate (AMP), niacinamide diphosphate (ADP), niacinamide adenosine triphosphate (ATP), niacinamide hydroquinone carboxylate, and combinations thereof.

A number of electron-donating compositions, such as huperzone, vinpocetin, vincamine, and yohimbine, are well known in the prior art for their memory enhancing benefits. Also, several proton-donating compositions of antioxidant group, such as glutathione, chlorogenic acid, rosmarinic acid, alpha-lipoic acid, ellagic acid, and such are also known to aid memory enhancement due to their antioxidant protection of brain cells and neurons. It has now been found that ion-pair compositions of such brain beneficial ingredients can be easily prepared, for example according to Equation 8, which are easier to formulate and provide synergistic brain beneficial benefits due to better bioavailability.

Glutathione+Huperzine→Huperzine Glutathionate (Equation 8)

The ion-pair compositions of the present invention can be made in pure form by a combination of required compositions, or they can be made in-situ in cosmetic or pharmaceutical compositions. Additionally, the alkali and alkaline earth metal derivatives of electron-accepting compositions, or mineral acid derivatives of electron-donating compositions can also be used for the preparation of ion-pair compositions of the present invention, as exemplified in Equation 9. A practical composition by this method is described in Example 4.

Sodium Ascorbate+Pyridoxine Hydrochloride→Pyridoxine Ascorbate+Sodium Chloride (Equation 9)

### EXAMPLES

The following examples are presented to illustrate presently preferred practice thereof. As illustrations they are not intended to limit the scope of the invention. All quantities are in weight %.

### Example 1

In-situ Preparation of Ion-Pair Composition of Glucosamine Ascorbate.

| | Column 1 | Column 2 |
|---|---|---|
| 1. Deionized Water | 33.2 | 33.2 |
| 2. Glucosamine Hydrochloride | 21.5 | 0.0 |
| 3. Sodium Ascorbate | 19.8 | 0.0 |
| 4. Propylene Glycol | 25.0 | 25.0 |
| 5. Glucosamine Ascorbate | 0.0 | 35.5 |
| 6. Geogard 221 (preservative) | 0.5 | 0.5 |
| 7. Sodium Chloride | 0.0 | 5.80 |

Procedure: All ingredients in Column 1 were mixed and heated at 40 to 50C. for 30 minutes. The product was cooled. A thin solution of composition in Column 2 was obtained, pH 5.6.

### Example 2

Preparation of Ion-Pair Niacinamide Ascorbate in a Pure Form.

Procedure: Niacinamide (6.0 grams) and Ascorbic Acid (9.0 grams) were mixed in distilled water (45 grams). A clear yellow solution was obtained. Water was then evaporated under vacuum. Niacinamide ascorbate (15.0 grams) was obtained as a yellow crystalline material.

### Example 3

Facial Cleanser Composition with Niacinamide Hydroxycitrate (16.5%, made by In-situ Process) for Facial Fat Reduction.

| | |
|---|---|
| (1) Glycerin | 38.3 |
| (2) Methyl paraben | 0.2 |
| (3) Hydroxycitric acid (HCA) | 10.4 |
| (4) Niacinamide | 6.1 |
| (5) Deionized Water | 19.1 |
| (6) Phenoxyethanol | 0.9 |
| (7) Sodium Cocoyl Isethionate | 20.0 |
| (8) Sodium Methyl Cocoyl Taurate | 5.0 |

Procedure: Mix deionized water, HCA, and niacinamide in a tank separately. A clear solution is obtained. All of the other ingredients are then added, except fragrance, and the mixture is heated and stirred at 60 to 70 degrees C. for about five to ten minutes until the mixture is homogenous. The homogeneous mixture is cooled to room temperature, and fragrance is added with mixing. A paste-like product is formed.

### Example 4

In-Situ Preparation of Niacin Hydroxycitrate from Niacin Hydrochloride and Tripotassium Hydroxycitrate.

(1) Tripotassium Hydroxycitrate Hydrate 3.4

(2) Deionized Water 95.3

(3) Niacin Hydrochloride 1.3

Procedure. Mix (1) to (3). Niacin Hydroxycitrate (3.96%) is formed in situ. Potassium chloride, also formed in this reaction, is precipitated by the addition of ethanol, followed by filtration.

### Example 5

The In-Situ Preparation of a 36.5% High Potency Benzyl Niacin Mandelate Anti-wrinkle Serum from Benzyl Niacin and Mandelic Acid.

| | Column 1 | Column 2 |
|---|---|---|
| (1) Mandelic Acid | 15.2 | 0.0 |
| (2) Benzyl Niacin | 21.3 | 0.0 |
| (3) Deionized Water | 30.0 | 30.0 |
| (4) Propylene Glycol | 34.5 | 34.5 |
| (5) Benzyl Niacin Mandelate | 0.0 | 36.5 |

Procedure: All ingredients in Column 1 were mixed and heated at 40 to 50 C. for 30 minutes. The product was cooled. A thin solution of composition in Column 2 was obtained.

### Example 6

Preparation of an Eye, Face, and Neck Gel with Niacinamide Salicylate, Niacinamide Glycolate, and Other Skin Beneficial Ingredients.

| | Column 1 | Column 2 |
|---|---|---|
| PEG-6 | 58.1 | 58.1 |
| Aristoflex AVC | 1.0 | 1.0 |
| Glycerin USP | 5.0 | 5.0 |
| Water | 20.0 | 20.32 |
| Geogard 221 (preservative) | 0.5 | 0.5 |
| Vitamin E Acetate | 0.5 | 0.5 |
| Niacinamide | 2.44 | 0.0 |
| Glycolic Acid (70%) | 1.08 | 0.0 |
| Salicylic Acid | 1.38 | 0.0 |
| Dimethicone | 4.0 | 4.0 |
| Dimethiconol | 4.0 | 4.0 |
| Cetyl Dimethiconol | 2.0 | 2.0 |
| Niacinamide Glycolate | 0.0 | 1.98 |
| Niacinamide Salicylate | 0.0 | 2.6 |

Procedure: Mix all ingredients in Column 1 and heat at 60 to 70 C. for 30 minutes. Cool to room temperature, and adjust pH to 4.5 with sodium hydroxide solution. A clear pale yellow gel of composition in Column 2 was obtained. The additional water in column 2 is from glycolic acid, which contains 30% water in it.

I claim:

1. The preparation of ion-pair cosmetic, pharmaceutical, or topical nutraceutical compositions in which;
(i) An electron accepting composition and an electron donating composition; or a proton accepting composition and a proton donating composition, are combined by an ion-pair bonding mode, and
(ii) Both electron donating and electron accepting compositions, or proton accepting and proton donating compositions, have skin and body beneficial benefits that are independent of each other, and
(iii) The ion-pair compositions that contain electron donating and electron accepting compositions, or proton accepting and proton donating compositions, undergo separation from each other to release such compositions in their original electronic compositional states when such ion-pair compositions reach physiological pH conditions.
(iv) A cosmetically or pharmaceutically acceptable carrier base composition that may contain additional skin, hair, and body beneficial compositions.

2. A composition according to claim 1, wherein electron accepting composition, or a proton accepting composition, can be selected from the group consisting of ascorbic acid, salicylic acid, lactic acid, glycolic acid, malic acid, mandelic acid, ascorbic acid, ascorbyl phosphoric acid, hydroxycitric acid, hydroxytetronic acid, citric acid, aleuritic acid, ellagic acid, rosmarinic acid, chlorogenic acid, polysulfonic acid, hyaluronic acid (HYA), Phytic Acid, Tartaric Acid, Hydroxytetronic Acid, Glucuronic Acid, Mucic Acid, Galacturonic Acid, Gluconic Acid, Saccharic Acid, Glucoheptonic Acid, alpha-Hydroxybutyric Acid, Tartronic Acid, alpha-Hydroxyisobutyric Acid, Isocitric Acid, alpha-Hydroxyisocaproic Acid, Dihydroxymaleic Acid, alpha-Hydroxyisovaleric Acid, Dihydroxytartaric Acid, beta-Hydroxybutyric Acid, Dihydroxyfumaric Acid, beta-Phenyllactic Acid, Atrolactic Acid, Galactonic Acid, Pantoic Acid, Glyceric Acid, and such. The quantities of such compositions can be safe and effective amounts as needed, and not limited to any specific limits.

3. A composition according to claim 1, wherein an electron donating composition, or a proton accepting composition can be selected from allantoin, arginine esters, lysine esters, cysteine esters, cystine esters, DNA, RNA, Adenosine, Guanosine, Cytosine, niacinamide, methyl niacin, benzyl niacin, lauryl niacin, pyridoxine, pyridoxal, pyridoxamine, yohimbine, vincamine, vinpocetin, huperzine, glucosamine, chitosan, chondroitin, glutathione, carnosine, creatine, creatinine, benzocaine, dibucaine, dyclonine, lidocaine, pramoxine, tetracaine, ephedrine, epinephrine, phenylephrine, cyclic or acyclic amines, amino acid esters, purines, pyrimidines, nucleic acids, nucleotides, DNA, RNA, peptide esters, guanidines, amidines, amino sugars, oxazoles, pyrroles, pyrazoles, imidazoles, pyridines, quinolines, quinazolines, quinoxalines, alkaloids, and their derivatives, and such. The quantities of such compositions can be safe and effective amounts as needed, and not limited to any specific limits.

4. A composition according to claim 1, wherein the ion-pair compositions can be selected from (or made in-situ from compositions indicated in the parentheses) Allantoin lactate (allantoin and lactic acid), allantoin glycolate (allantoin and glycolic acid), allantoin mandelate (allantoin and mandelic acid), allantoin malate (allantoin and malic acid), allantoin ascorbate (allantoin and ascorbic acid), allantoin phytate (allantoin and phytic acid), allantoin citrate (allantoin and citric acid), allantoin hydroxy citrate (allantoin and hydroxy citric acid), allantoin aleurate (allantoin and aleuritic acid), allantoin salicylate (allantoin and salicylic acid), allantoin hyaluronate (allantoin and hyaluronic acid), glucosamine lactate (glucosamine and lactic acid), glucosamine glycolate (glucosamine and glycolic acid0, glucosamine malate (glucosamine and malic acid), glucosamine mandelate (glucosamine and mandelic acid), glucosamine ascorbate (glucosamine and ascorbic acid, glucosamine phytate (glucosamine and phytic acid), glucosamine citrate (glucosamine and citric acid), glucosamine hydroxy citrate (glucosamine and hydroxy citric acid), glucosamine aleurate (glucosamine and aleuritic acid), glucosamine salicylate (glucosamine and salicylic acid), glucosamine hyaluronate (glucosamine and hyaluronic acid), creatine lactate (creatine and lactic acid), creatine glycolate (creatine and glycolic acid), creatine malate (creatine and malic acid), creatine mandelate (creatine and mandelic acid), creatine ascorbate (creatine and ascorbic acid), creatine phytate (creatine and phytic acid), creatine citrate (creatine and citric acid), creatine hydroxy citrate (creatine and hydroxy citric acid), creatine aleurate (creatine and aleuritic acid), creatine salicylate (creatine and salicylic acid), creatine hyaluronate (creatine and hyaluronic acid), niacinamide lactate (niacinamide and lactic acid), niacinamide glycolate (niacinamide and glycolic acid, niacinamide malate (niacinamide and malic acid), niacinamide mandelate (niacinamide and mandelic acid), niacinamide ascorbate (niacinamide and ascorbic acid), niacinamide phytate (niacinamide and phytic acid), niacinamide citrate (niacinamide and citric acid), niacinamide hydroxy citrate (niacinamide and hydroxy citric acid), niacinamide aleurate (niacinamide and aleuritic acid), niacinamide salicylate (niacinamide and salicylic acid), niacinamide hyaluronate (niacinamide and hyaluronic acid), pyridoxine lactate (pyridoxine and lactic acid), pyridoxine glycolate (pyridoxine and glycolic acid), pyridoxine malate (pyridoxine and malic acid), pyridoxine mandelate (pyridoxine and mandelic acid), pyridoxine ascorbate (pyridoxine and ascorbic acid, pyridoxine phytate (pyridoxine and phytic acid, pyridoxine citrate (pyridoxine and citric acid), pyridoxine hydroxy citrate (pyridoxine and hydroxy citric acid), pyridoxine aleurate (pyridoxine and aleuritic acid), pyridoxine salicylate (pyridoxine and salicylic acid), pyridoxine hyaluronate (pyridoxine and hyaluronic acid), chitosan lactate, chitosan glycolate, chitosan malate, chitosan mandelate, chitosan ascorbate, chitosan phytate, chitosan citrate, chitosan hydroxy citrate, chitosan aleurate, chitosan salicylate, chitosan hyaluronate and combinations thereof.

5. A composition according to claim 1, wherein skin beneficial cosmetic or drug base composition can be additionally selected to provide treatment of skin aging, skin wrinkles reduction, skin exfoliating, treatment of acne, treatment of rosacea, age-spots reduction, skin surface whitening, skin surface brightening, striae distensae (stretch marks) reduction, treatment of pimples, treatment of skin infections and lesions, varicose and spider veins reduction, blood microcirculation improvement, UVA/UVB protection of skin, skin redness reduction benefits, antimicrobial agents, antifungal agents, Vitamins and provitamins, hormones, peptides, skin protectants, excess topical fat reduction, cellulite control, skin and body toning benefits, or combinations thereof.

6. The compositions according to claim 1, wherein the cosmetically or pharmaceutically acceptable carrier base can be traditional water and oil emulsions, suspensions, colloids, microemulsions, clear solutions, suspensions of nanoparticles, emulsions of nanoparticles, or anhydrous compositions.

7. A composition according to claim 1, wherein cosmetically or pharmaceutically acceptable carrier base can optionally include additional skin beneficial ingredients selected from skin cleansers, surfactants (cationic, anionic, non-ionic, amphoteric, and zwitterionic), skin and hair conditioning agents, vitamins, hormones, minerals, plant extracts, anti-inflammatory agents, concentrates of plant extracts, emollients, moisturizers, skin protectants, humectants, silicones, skin soothing ingredients, analgesics, skin penetration enhancers, solubilizers, moisturizers, emollients, alkaloids, memory enhancers, anesthetics, colorants, perfumes, preservatives, seeds, broken seed nut shells, silica, clays, beads, luffa particles, polyethylene balls, mica, pH adjusters, processing aids, and combinations thereof. The quantities of such ingredients can be safe and effective amounts as needed, and not limited to any specific limits.

8. A composition according to claim 7, wherein the composition to promote excess fat reduction, cellulite control, or toning benefits can be selected from the group consisting of Forskohlin extract (from Coleus forskohlii plant), Hydroxycitric acid, (from Garcinia cambogia, and plants of Garcinia family), L-Carnitine, Creatine, Human growth hormone (HGH), Chromium picolinate, Kola seed extract, Caffeine, Niacinamide, Psyllium husk, Chitosan, Lipoprotein complexes, Polyphenols, Gymnemic acid, Pyruvic acid and Pyruvate salts, salts of Hydroxycitric acid, Phaseolamin (from Phaseolus vulgaris extract), DHEA, Chitosan, Theophylline, Theobromine (or salts thereof such as Aminophylline), Roselle tea extract, Arabinose, Inosine, Adenosine, Fructose-1,6-diphosphate, Adenosine triphosphate (ATP), Adenosine diphosphate (ADP), Indomethacin, Baicalein, Extract of the plant of genus Tephrosia, Natriuretic peptide, Laminaria extract, Extract from berries of Panax genus plant, Gymnema sylvestre extract, 9-cis, 11-trans Conjugated linoleic acid and 10-trans, 12-cis conjugated linoleic acid isomers (conjugated linoleic acid, CLA), Synephrine, Hordenine, Octopamine, Tyramine, N-Methyltyramine, Azaftig, Extract of Climbing ivy (Hedera helix), Extract of Arnica (Arnica montana), Extract of Rosemary (Rosmarinus officinalis), Extract of Marigold (Calendula officinalis), Extract of Sage (Salvia officinalis), Extract of Ginseng (Panax ginseng), Extract of St. Johns-wart (Hypericum perforatum), Extract of Ruscus (Ruscus aculeatus), Extract of meadowsweet (Filipendula ulmaria), Extract of Orthosiphon (Ortosifon stamincus), and combinations thereof.

9. A composition according to claim 7, wherein the cosmetic or drug ingredient that can be selected from a group consisting of antioxidants, collagen and elastin synthesis boosters, various hydroxy acids (alpha hydroxy acids, beta hydroxy acids, and polyhydroxy acids), vitamins, hormones, skin whitening agents, UVA/UVB sunscreens, antimicrobial agents, antifungal agents, blood microcirculation improvement agents (vasodilatory or vasoconstrictive), skin protectant drug actives, and combinations thereof. The quantities of such compositions can be safe and effective amounts as needed, and not limited to any specific limits.

10. A composition according to claim 7, wherein the composition to promote collagen and elastin in the skin can be selected from Ascorbic acid, Ascorbic acid derivatives, Glucosamine ascorbate, Arginine ascorbate, Lysine ascorbate, Glutathione ascorbate, Nicotinamide ascorbate, Niacin ascorbate, Allantoin ascorbate, Creatine ascorbate, Creatinine ascorbate, Chondroitin ascorbate, Chitosan ascorbate, DNA Ascorbate, Carnosine ascorbate, Vitamin E, various Vitamin E derivatives, Tocotrienol, Rutin, Quercetin, Hesperedin (Citrus sinensis), Diosmin (Citrus sinensis), Mangiferin (Mangifera indica), Mangostin (Garcinia mangostana), Cyanidin (Vaccinium myrtillus), Astaxanthin (Haematococcus algae), Lutein (Tagetes patula), Lycopene (Lycopersicum esculentum), Resveratrol (Polygonum cuspidatum), Tetrahydrocurcumin (Curcuma longa), Rosmarinic acid (Rosmarinus officinalis), Hypericin (Hypericum perforatum), Ellagic acid (Punica granatum), Chlorogenic acid (Vaccinium vulgaris), Oleuropein (Olea europaea), alpha-Lipoic acid, Niacinamide lipoate, Glutathione, Andrographolide (Andrographis paniculata), Carnosine, Niacinamide, Potentilla erecta extract, Polyphenols, Grapeseed extract, Pycnogenol (Pine Bark extract), and combinations thereof. The quantities of such compositions can be safe and effective amounts as needed, and not limited to any specific limits.

11. A composition according to claim 7, wherein the skin beneficial hydroxy acid can be selected from the group consisting of salicylic acid, lactic acid, glycolic acid, malic acid, mandelic acid, ascorbic acid, ascorbyl phosphoric acid, hydroxycitric acid, hydroxytetronic acid, citric acid, aleuritic acid, ellagic acid, rosmarinic acid, chlorogenic acid, polysulfonic acid, and hyaluronic acid (HYA). The quantities of such compositions can be safe and effective amounts as needed, and not limited to any specific limits.

12. A composition according to claim 7, wherein the skin beneficial skin whitening agent can be selected from hydroquinone, arbutin, hydroquinone derivatives, Paper Mulberry extract (Broussonetia kazinoke), Mitracarpe extract (Mitracarpus scaber), Bearberry extract (Arctostaphylos uva ursi), Yellow Dock extract (Rumex crispus and Rumex occidentalis), Glutathione, Leucocyte extract, Aspergillus orizae extract (Aspergillus orizae), Licorice Root extract (Glycyrrhiza glabra), Rosmarinic acid (Rosmarinus officinalis), Tetrahydrocurcumin, Green Tea extract (Camellia sinensis), Yohimbe extract (Pausinystalia yohimbe), Ecklonia cava extract, niacinamide, Hydroxytetronic acid, Spondias mombin extract, Maprounea guianensis extract, Walteria indica extract, Gouania blanchetiana extract, Cordia schomburgkii extract, Randia armata extract, Hibiscus furcellatus extract, and combinations thereof. The quantities of such compositions can be safe and effective amounts as needed, and not limited to any specific limits.

13. A composition according to claim 7, wherein the skin beneficial antioxidant composition can be selected from Ascorbic acid, Ascorbic acid derivatives, Vitamin E, Vitamin E derivatives, Tocotrienol, Rutin, Quercetin, Hesperedin (Citrus sinensis), Diosmin (Citrus sinensis), Mangiferin (Mangifera indica), Mangostin (Garcinia mangostana), Cyanidin (Vaccinium myrtillus), Astaxanthin (Haematococcus algae), Lutein (Tagetes patula), Lycopene (Lycopersicum esculentum), Resveratrol (Polygonum cuspidatum), Tetrahydrocurcumin (Curcuma longa), Rosmarinic acid (Rosmarinus officinalis), Hypericin (Hypericum perforatum), Ellagic acid (Punica granatum), Chlorogenic acid (Vaccinium vulgaris), Oleuropein (Olea europaea), alpha-Lipoic acid, Glutathione, Andrographolide, Grapeseed extract, Green Tea Extract, Polyphenols, Pycnogenol (Pine Bark extract), White Tea extract, Black Tea extract, (Andrographis paniculata), Carnosine, Niacinamide, Emblica extract, and combinations thereof. The quantities of such compositions can be safe and effective amounts as needed, and not limited to any specific limits.

14. A composition according to claim 7, wherein the UVA/UVB sunscreen composition can be selected from Titanium dioxide, Zinc oxide, Galanga extract (Kaempferia galanga), Benzophenone-3, Benzophenone-4, Ethylhexyl Methoxycinnamate, Homosalate, Ethylhexyl salicylate, Octocrylene, Menthyl anthranilate, Avobenzone, Lawsone, Sulisobenzone, Trolamine salicylate, Lawsone, Glyceryl aminobenzoate, Cinoxate, and PABA. and combinations thereof. The quantities of such compositions can be safe and effective amounts as needed, and not limited to any specific limits.

15. A Composition According to claim 7, wherein the blood microcirculation improvement composition can be selected from Horse Chestnut Extract (Aesculus hippocastanum extract)), Esculin, Escin, Yohimbine, Capsicum Oleoresin, Capsaicin, Niacin, Niacin Esters, Methyl Nicotinate, Benzyl Nicotinate, Ruscogenins (Butchers Broom extract; Ruscus aculeatus extract), Diosgenin (Trigonella foenumgraecum, Fenugreek), Emblica extract (Phyllanthus emblica extract), Asiaticoside (Centella asiatica extract), Boswellia Extract (Boswellia serrata), Ginger Root Extract (Zingiber Officianalis), Piperine, Vitamin K, Melilot (Melilotus officinalis extract), Glycyrrhetinic acid, Ursolic acid, Sericoside (Terminalia sericea extract), Darutoside (Siegesbeckia orientalis extract), Amni visnaga extract, extract of Red Vine (Vitis-Vinifera) leaves, apigenin, phytosan, luteolin, and combinations thereof. The quantities of such compositions can be safe and effective amounts as needed, and not limited to any specific limits.

16. A composition according to claim 7, wherein the antimicrobial composition can be selected from Berberine, Triclosan, Triclocarban, various Tritons (quaternary ammonium compounds), Benzyl Alcohol, Dehydroacetic Acid, Phenoxyethanol, and combinations thereof. The quantities of such compositions can be safe and effective amounts as needed, and not limited to any specific limits.

17. A composition according to claim 7, wherein the vitamin composition can be selected from Vitamin A, members of Vitamins B group, Vitamin C, Vitamin D, Vitamin E, Vitamin K, Carotenes, Biotin, Folic Acid, and combinations thereof. The quantities of such ingredients can be safe and effective amounts as needed, and not limited to any specific limits.

18. A composition according to claim 7, wherein the hormone composition can be selected from progesterone, androsterone, dehydroepiandrosterone (DHEA), Pregnenolone, androstenedione, melatonin, testosterone, and combinations thereof. The quantities of such compositions can be safe and effective amounts as needed, and not limited to any specific limits.

19. A composition according to claim 7, wherein the skin protectant drug composition can be selected from Allantoin, petrolatum, glycerin, dimethicone, urea, calamine, cocoa butter, kaolin, zinc acetate, zinc carbonate, and combinations thereof. The quantities of such compositions can be safe and effective amounts as needed, and not limited to any specific limits.

20. A composition according to claim 7, wherein the memory enhancement composition can be selected from various alkaloids including huperzine, vincamine, vinpocetin, and yohimbine.
